(19) United States
(12) Patent Application Publication
(10) Pub. No.: US 2009/0112238 A1
(43) Pub. Date: Apr. 30, 2009

(54) FISTULA BRUSH DEVICE

(75) Inventors: Timothy W. Pitts, Bloomington, IN (US); Sheila L. Hieber, Bloomington, IN (US); John H. Ward, Spencer, IN (US)

Correspondence Address: BRINKS HOFER GILSON & LIONE/CHICAGO/COOK, PO BOX 10395, CHICAGO, IL, 60610, US

(73) Assignee: Vance Products Inc., d/b/a Cook Urological Inc., Spencer, IL (US)

(21) Appl. No.: 11/925,362

(22) Filed: Oct. 26, 2007

(51) Int. Cl. A61B 17/22

(52) U.S. Cl. 606/159

(57) ABSTRACT

A medical device includes an elongate member extending between a first end portion and a second end portion and a brush disposed upon the elongate member between the first end portion and the second end portion. A first aperture is disposed proximate the first end portion and a second aperture disposed proximate the second end portion. A first suture is disposed through the first aperture and tied upon itself to form a first loop, and a second suture is disposed through the second aperture and tied upon itself to form a second loop, wherein each of the first loop and the second loop are configured to receive an implantable material.

### BACKGROUND

A variety of fistulae are known to occur in humans and other mammals. These fistulae can occur for a number of reasons, such as but not limited to, congenital defects, as a result of an inflammatory bowel disease, such as Chron's disease, irradiation, trauma, childbirth, or as a side effect of a surgical procedure. Further several different types of fistulae can occur, for example, urethra-vaginal fistulae, vesico-vaginal fistulae, tracheo-esophageal fistulae, gastro-cutaneous fistulae, and any number of anorectal fistulae, such as recto-vaginal fistulae, recto-vesical fistulae, recto-urethral fistulae, or recto-prostatic fistulae.

The field of the invention is that of medical and surgical instruments, and in particular, medical and surgical instruments intended primarily for minimally-invasive procedures such as procedures to correct various types of fistulae.

### BRIEF SUMMARY

The first aspect of the present invention provides a medical device. The medical device includes an elongate member extending between a first end portion and a second end portion and a brush disposed upon the elongate member between the first end portion and the second end portion. A first aperture is disposed proximate the first end portion and a second aperture is disposed proximate the second end portion. A first suture is disposed through the first aperture and tied upon itself to form a first loop, and a second suture is disposed through the second aperture and tied upon itself to form a second loop, wherein each of the first loop and the second loop are configured to receive an implantable material.

A second aspect of the present invention provides a medical device. The medical device includes an elongate member extending between a first end portion and a second end portion, the elongate member defining a lumen through at least a portion thereof. A brush is disposed upon the elongate member between the first end portion and the second end portion. A first aperture is defined in the first end portion and a second aperture is defined in the second end portion. A first suture is disposed through the first aperture and tied to itself to define a first loop and a second suture disposed through the second aperture and tied to itself to define a second loop, wherein each of the first and second loops are configured to receive an implantable material.

Advantages of the present invention will become more apparent to those skilled in the art from the following description of the preferred embodiments of the invention that have been shown and described by way of illustration. As will be realized, the invention is capable of other and different embodiments, and its details are capable of modification in various respects. Accordingly, the drawings and description are to be regarded as illustrative in nature and not as restrictive.

### BRIEF DESCRIPTION OF THE DRAWINGS

FIG. 1 is a perspective view of an elongate device configured to debride a fistula and insert an implantable material therein.

FIG. 2 is a side view of another elongate device configured to debride a fistula and insert an implantable material therein.

FIG. 3 is a side view of a modified device of FIG. 2.

FIG. 4 is the device of FIG. 2 with an implantable device installed thereupon.

FIG. 5 is a perspective view of an implantable material for use with the elongate device.

### DETAILED DESCRIPTION OF THE DRAWINGS AND THE PREFERRED EMBODIMENTS

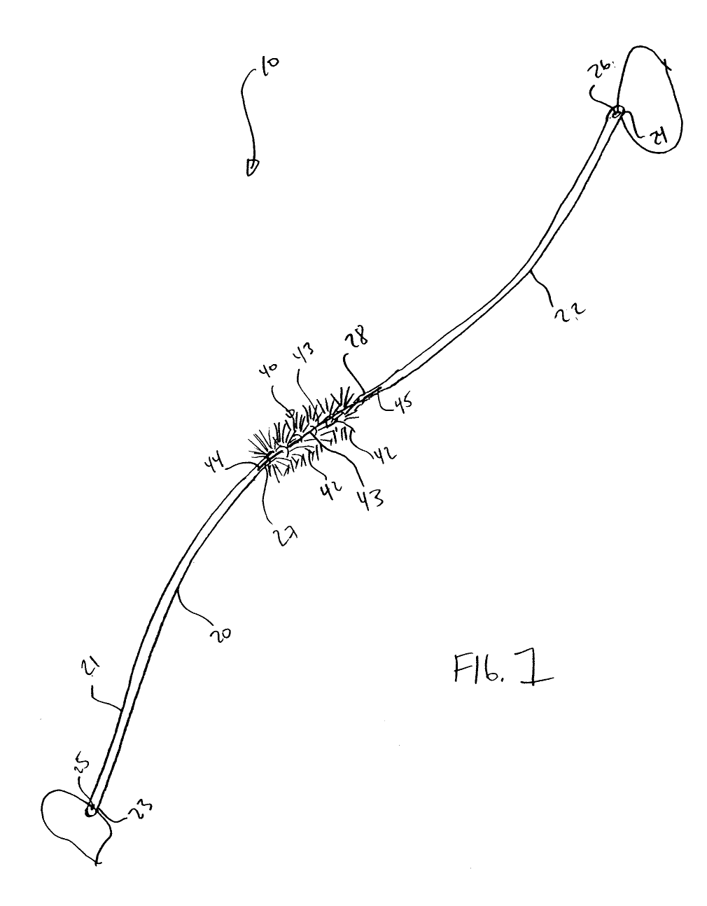

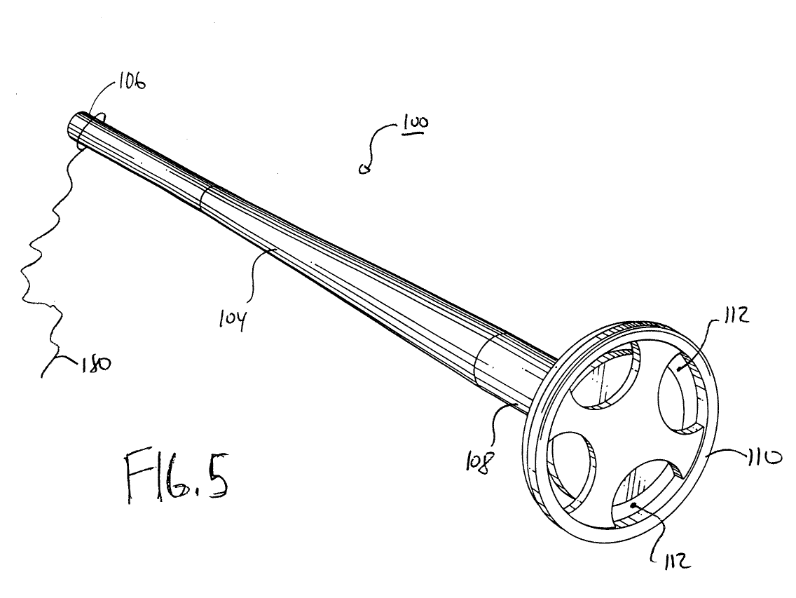

Turning now to the embodiments shown in FIGS. 1-5, a device 10 for treating and repairing fistulae is provided. The device 10 includes first and second elongate members 21, 22 and a brush portion 40 that is engaged to a proximal end 27, 28 of each of the first and second elongate members 21, 22. The brush portion 40 includes a plurality of bristles 42 that extend radially from a wire 43, which extend along the length of the brush portion 40. The plurality of bristles 42 may be disposed upon the wire 43 in a plurality of clumps 42a that are each disposed along a portion of the length of the wire 43.

The plurality of clumps 42a each include a plurality of bristles 42 that extend radially from substantially the same point of the wire 43, with sufficient bristles 42 provided such that bristles 42 extend from the wire 43 in substantially every radial direction along the outer circumference of that portion of the wire 43. In some embodiments, the brush portion 40 may include between about 6 and about 12 clumps 42a of bristles 42. In some embodiments, the brush portion 40 may include 6, 8, 10, 12 or another number of clumps 42a of bristles 42 along a portion of the length of the wire 43. The clumps 42a may be substantially evenly spaced between neighboring clumps 42a along a portion of the length of the wire 40 or the clumps 42a may be spaced at varying distances from neighboring clumps 42a along the length of a portion of the wire 40.

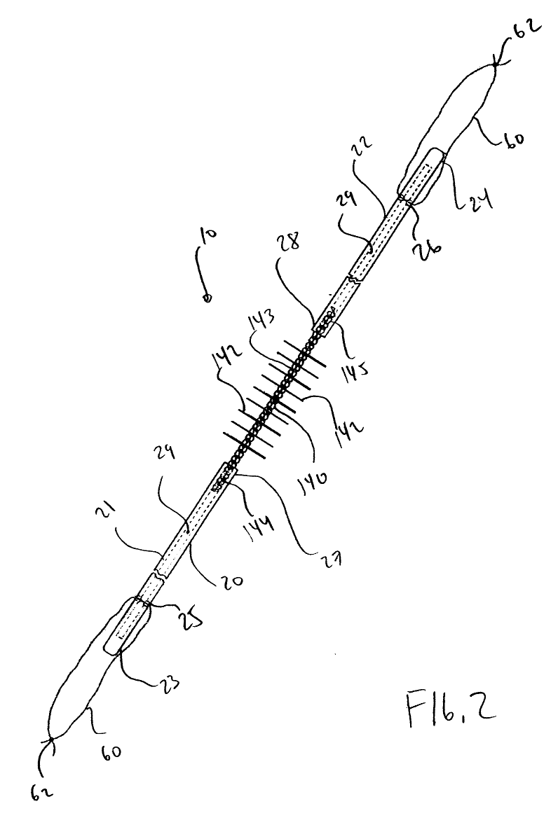

As shown in FIG. 1, the wire 43 may be a single wire that extends between a first end 44 and a second end 45 and receives the plurality of bristles 42 within a central portion 46 of the wire 43. In other embodiments shown in FIGS. 2-4, the wire 43 may be formed from a plurality of wires 43b that are woven or otherwise engaged together to form a single member.

In other embodiments shown in FIGS. 2-4, a brush portion 140 is provided on the device 10 and includes a plurality of bristles 142 that are retained by a plurality of discrete wires 143b woven or otherwise fixed together to form a bundle of wires 143. In other embodiments the three or more discrete wires 143b may be woven or otherwise engaged together to form the bundle of wires 143. In embodiments where two or more wires 143b are woven together to form the bundle of wires 143, the plurality of bristles 142 are retained upon the bundle of wires 143 with a proximal end of each bristle 142 being retained between the neighboring wires 143b and retained thereupon due to the tightly woven nature of the multiple wires 143b.

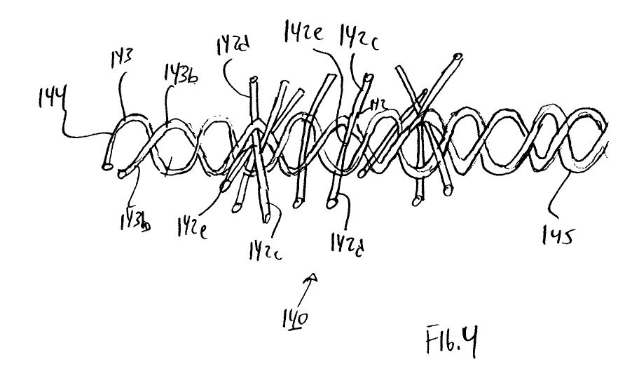

In some embodiments and as shown in FIG. 4, the plurality of bristles 142 may be each formed with a first portion 142c and a second portion 142d, wherein the first and second portions 142c, 142d each extend radially from the bundle of wires 143. The bristles 142 each additionally include a central portion 142e between the first and second portions 142c, 142d that is retained between neighboring wires 143b. With the central portion 142e retained by the bundle of wires 143, the first and second portions 142c, 142d extend radially from the bundle of wires 143 in opposing or different directions. In some embodiments, several bristles 142 are retained at substantially the same position along the length of the bundle of wires 143 such that the first and second ends 142c, 142d of each bristle 142 extends radially from substantially the same position along the length of the bundle of wires 143 to form a clump 142a of bristles 142. Similar to the embodiments discussed above, the brush portion 140 may include a plurality of clumps 142a of bristles 142, such as between 6 and 12 clumps 142a of bristles 142. In some embodiments 6, 8, 10, 12 or other numbers of clumps 142a of bristles 142 may be disposed along the length of the brush portion 140.

The first and second elongate members 21, 22 are each configured with an extended distal end 23, 24 and a proximal end 27, 28. The elongate members 21, 22 each additionally include a lumen 29 defined through at least a portion of the elongate member 21, 22. In some embodiments, the lumen 29 extends to a position proximate the distal end 23, 24 along the length of the elongate member 21, 22 to a proximal end 27, 28 of each elongate member 21, 22. Specifically, the lumen 29 in each elongate member 21, 22 allows for fluid or gaseous communication from the proximal end 27, 28 of each elongate member 21, 22 to a location proximate the distal end 23, 24 of each elongate member 21, 22.

The proximal end 27, 28 includes an aperture 27a, 28a defined within the end surface of the respective proximal end 27, 28. The apertures 27a, 28a are each configured to receive one of the first or second ends 44, 45 (or in other embodiments 144, 145) of the brush portion 40 (140) therein to fix the brush portion 40 (140) to the respective elongate member 21, 22. Specifically, the lumen 29 and respective aperture 27a, 28a may have an inner diameter slightly larger than the outer diameter of the first and second ends 44, 45 (144, 145) of the brush portion 40 (140). The first and second ends 44, 45 (144, 145) may be retained within the respective first and second apertures 27a, 28a with an adhesive, such as cyanoacrylate. In other embodiments, the lumen 29 and the first and second apertures 27a, 28a have an inner diameter that is substantially the same as the outer diameter of the first and second ends 44, 45 (144, 145) of the brush portion 40 (140) to allow the respective first and second end 44, 45 (144, 145) to be retained within the lumen 29 to rigidly connect the brush portion 40 (140) to the first and second elongate members 21, 22.

In some embodiments, the first and second elongate members 21, 22 may be made from a substantially flexible and compliant material such as nylon or other types of relatively soft plastic. Similarly, the wire 43 (or bundle of wires 143) may be made from a much harder and less compliant material such as stainless steel. In these embodiments, the wire 43 (or bundle of wires 143) may be formed with a slightly larger outer diameter than the inner diameter of the apertures 27a, 28a and the lumen 29 of the first and second elongate members 21, 22. Accordingly, when one of the first or second ends 44, 45 (144, 145) of the brush portion 40 (140) is inserted into the respective aperture 27a, 28a, the end of the brush portion 40 (140) causes the respective elongate member 21, 22 to slightly deform to accept the slightly larger end 44, 45 (144, 145) of the brush portion 40 (140). The slight deformation of the elongate member 21, 22 increases the strength of the connection between the brush portion 40 (140) and the elongate members 21, 22, which increases the overall strength of the device 10.

The first and second members 21, 22 are each made from a relatively flexible material, which allows the first and second members 21, 22 to each be elastically deformed by the user to allow the device 10 to be inserted into the patient as discussed below.

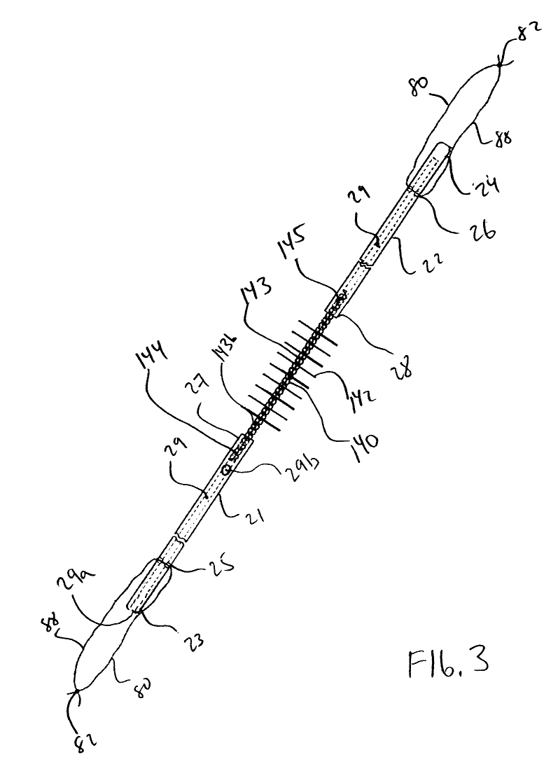

In some embodiments shown in FIG. 3, the lumen 29 may extend the length of each elongate member 21, 22 between an aperture 23a, 24a on the respective distal end portion 23, 24 of the elongate member 21, 22 and the proximal aperture 27a, 28a. The lumen 29 allows for fluid or gaseous communication along the entire length of the elongate member 21, 22. In some embodiments, the distal end portion 23, 24 may be configured to accept a Luer lock. In the embodiment shown in FIG. 3, a radial aperture 29b may be provided proximate the proximal end portion 27, 28 of the respective elongate member 21, 22 and provide a path for fluid or gas entering the lumen 29 through the respective distal aperture 23a, 24a to leave the lumen 29. The radial aperture 29a is configured to allow fluid or gas present within the lumen 29 to leave the elongate member 21, 22 through the radial aperture 29a.

The radial aperture 29a may be defined within the proximal end portion 27, 28 of the respective elongate member 21, 22 such that the respective first or second end 44, 45 (144, 145) of the brush portion 40 (140) does not extend through the lumen 29 far enough to block the radial aperture 29a.

A length of suture material 88 is provided in combination with one or both of the elongate members 21, 22. Specifically, the length of suture material 88 is threaded through a distal aperture 25, 26 defined through opposing side surfaces of the elongate member 21, 22 proximate to the distal end portion 23, 24 of the respective elongate member 21. In some embodiments, the distal aperture 25, 26 extends through the entire cross-section of the respective elongate member 21, 22, while in embodiments where the lumen 29 extends through the entire length of the elongate member 21, 22 the distal aperture 25, 26 extends through opposing portions of the elongate member (i.e. those opposing portions defining the lumen 29) and the lumen 29. Regardless of the structure of the distal aperture 25, 26 with respect to the lumen 29 and the remainder of the elongate member 21, 22, the distal aperture 25, 26 is configured to allow the suture material 88 to extend through the elongate member 21, 22 and therefore be retained by the elongate member 21, 22.

The suture material 88 includes two opposite ends that are tied together with a knot 82 to form a loop 80 after the suture material 88 is threaded through the distal aperture 25, 26. The loop 80 (and the length of the suture material 88 provided to form the loop 80) is configured to such that a portion of the loop 80 extends beyond the extreme distal end of the elongate member 21, 22. The loop 80 is configured to receive an implantable material 100 thereupon either directly or through a second suture 180 that is tied to both the implantable material and the loop of the suture material 88. The implantable material 100 is configured to be disposed and retained within the fistula to substantially block fluid or gaseous communication through the fistula between the rectum and the vagina, and provide a support structure or scaffold material for the patient's tissue to grow or redistribute thereupon to permanently block and close the fistula.

A plug, chunk, length, or portion of an implantable material 100 is provided and configured to be removeably connected to one or more suture loops 80 by way of a second length of suture material 180 that is tied to a portion of the implantable material. Several different suitable designs and structures for the implantable material 100 configured for use with the device 10 are disclosed and described in U.S. Publication No. 2007/0198059, filed on Jan. 31, 2007, U.S. Provisional Application No. 60/947,573, filed on filed on Jul. 2, 2007, and U.S. Provisional Application No. 60/815,802, filed on Jun. 21, 2006 and subsequently filed as PCT Application No. PCT/US07/71798 on Jun. 21, 2207, which are each assigned to a subsidiary of the assignee of this application and the entirety of which are each fully incorporated by reference herein. The second suture material 180 may be tied to the loop 80 of suture material 88 to connect the implantable material 100 to the device 10.

The implantable material 100 may include an elongate portion 104 that is of a length suitable for extending through a typical rectovaginal fistula, which is normally between approximately 5 mm and 10 mm but which may be smaller or larger depending on the patient's anatomy. In some embodiments, the elongate portion may be between about 5 and 10 cm in length, to provide an elongate portion 104 of sufficient length to extend from the fistula after placement and allow the elongate portion 104 to be sewn to the patient's vaginal mucosa for retention within the patient. The elongate portion 104 of the implantable device 100 may be formed with a plurality of different diameters to be used with fistulae of different sizes and shapes, to allow the elongate portion 104 to be inserted and drawn through the fistula tract while substantially enclosing the aperture between the rectum and the vagina defined by the fistula. The second suture material 180 may be tied to a distal end 106 of the elongate portion 104, which is then tied to one of the loops 80 on an elongate portion 21, 22 of the device 10.

The implantable device 100 may additionally include a button 110 that is fixed to a proximal end 108 of the elongate portion 104 with a suture material. The button 110 may be substantially circular or formed with other circumferential shapes. The button 110 provides a retention structure that does not fit within the fistula and a surface that is configured to be attached to the mucosa forming the rectal wall neighboring the fistula. The button 110 may include one or more apertures 112 (in some embodiments two or four apertures 112) that provide suitable locations upon the button 110 for the physician to sew to the rectal mucosa proximate the fistula. The button 110 may be made from a substantially rigid implantable material such as plastic, or in other embodiments may be made from a biological material similar to that used to make the elongate portion 104 as discussed below. In some embodiments, the button 110 is not reabsorbed and separates from the elongate portion 104 of the implantable device 100 after some time has elapsed after implantation due to the connection between the elongate portion 104 and the rectal mucosa disintegrating.

The implantable device 100 is configured for ultimate placement within the fistula to promote tissue growth proximate and into the aperture defined by the fistula to close or occlude the fistula. The elongate portion 104 of the implantable material 100 may be made from an extracellular matrix (ECM) material, such as submucosa, renal capsule membrane, dura mater, pericardium, serosa, peritoneum, or basement membrane. A preferred elongate portion 104 may be submucosa, such as submucosa derived from a warm-blooded vertebrate. Mammalian submucosa materials are preferred. In particular, submucosa materials derived from animals raised for meat or other product production, e.g. pigs, cattle, or sheep, may be advantageous. Porcine submucosa provides a particularly preferred material, especially porcine small intestine submucosa (SIS), more especially porcine small intestine submucosa retaining substantially its native cross-linking.

The submucosa or other ECM material can be derived from any suitable organ or other biological structure, including for example submucosa derived from the alimentary, respiratory, intestinal, urinary or genital tracts of warm-blooded vertebrates. Submucosa useful in the present invention can be obtained by harvesting such tissue sources and delaminating the submucosa from smooth muscle layers, mucosal layers, and/or other layers occurring in the tissue source.

As prepared, the extracellular matrix material may optionally retain growth factors or other bioactive components native to the source tissue. For example, the matrix material may include one or more growth factors such as basic fibroblast growth factor (FGF-2), transforming growth factor beta (TGF-beta), epidermal growth factor (EGF), and/or platelet derived growth factor (PDGF). As well submucosa or other ECM material may include other biological materials such as heparin, heparin sulfate, hyaluronic acid, fibronectin and the like. Thus, generally speaking, the ECM material may include a bioactive component that induces, directly or indirectly, a cellular response such as a change in cell morphology, proliferation, growth, protein, or gene expression. Further, in addition or as an alternative to the inclusion of such native bioactive components, non-native bioactive components such as those synthetically produced by recombinant technology, or other methods, may be incorporated into the ECM material.

ECM material used as the elongate portion 104 of the implantable material 100 may be highly purified, for example, as described in U.S. Pat. No. 6,206,931, the disclosure thereof is fully incorporated by reference herein. For additional information concerning submucosa and its isolation, purification, and treatment, reference can be made, for example, to U.S. Pat. Nos. 4,902,508, 5,554,389, 5,993,844, 6,206,931, 6,099,567, and U.S. Published Application Nos. 2004/0180042, 2006/0201996, and 2007/0166395.

The device 10 is sized, shaped, and configured to be inserted into the patient to control and correct a rectovaginal fistula. In use the physician identifies one or more fistulae openings in one or both of the rectum or the vagina and inserts the distal end portion 23, 24 of one of the elongate portions 21, 22 into the most convenient and accessible fistula opening (i.e. either inserted into the fistula from the rectal or the vaginal side of the fistula). The device 10 is threaded through the fistula opening and navigated through the fistula wall until the distal end portion 23, 24 of the leading elongate portion 21, 22 extends out of the other of the openings in the rectum or vagina. Because the device is symmetrical about the brush portion 40 (140) (i.e. the first and second elongate portions 21, 22 are substantially the same), the physician may insert either elongate portion 21, 22 into either the rectal or vaginal fistula openings as convenient. The device 10 then translated through the fistula until the brush portion 40 (140) is proximate and extends through the fistula.

The physician then reciprocatingly translates at least a portion of the brush portion 40 (140) through the fistula in both directions to agitate or debride the inner circumferential surface of the fistula by contact between the plurality of bristles 42 of the brush portion 40 and the fistula wall. The reciprocating agitation aids in the removal of any dead or unstable tissue therefrom to assist in the application, receipt, acceptance, and retention of the elongate portion 104 of the implantable material 100 within the fistula. Specifically, the mechanical agitation of the inner surface of the fistula aids in the removal of any dead or unhealthy tissue bordering the fistula tract, which allow the elongate portion 104 of the implantable material 100 to contact healthier tissue after insertion.

After the fistula tissue is agitated or debrided, the fistula may be flushed with sterile saline or similar solutions to clean any fully or partially removed tissue from the fistula. In some embodiments, the fistula may additionally be flushed with a pharmacology active fluid to achieve a desired pharmacological response in the neighboring tissue, or a sclerosant or sealant to block a portion of the fistula if necessary. In other embodiments, the fistula tract may additionally be flushed with a liquid configured to aid in the removal of dead or damaged tissue defining the fistula tract. In some embodiments, the fistula is flushed by the physician with a bulb syringe or similar device that is positioned proximate to the most convenient opening of the fistula. In some embodiments such as the embodiment shown in FIG. 3, the fistula tract is flushed with fluid that flows through the lumen 29 which one of the elongate members 21, 22 and enters the fistula (and leaves the device 10) through a radial lumen 29a disposed proximate the brush portion 40 (140).

After the physician has flushed the fistula and the circumferential tissue within the fistula, the implantable material 100 is affixed to the suture loop 80 that extends through an aperture 25, 26 in the elongate member 21, 22 that extends from the patient's rectum. In some embodiments, a second suture 180 is attached to a distal end 106 of the elongate member 104 of the implantable material 100. The opposite end of the second suture 180 is tied to the loop 80 on the elongate member 21, 22 that extends from the patient's rectum.

The physician then with draws the device 10 from the patient's vagina, which pulls the brush portion 40 (140) from the fistula, and then pulls the elongate member 21, 22 that extended from the rectum through the fistula and eventually through the patient's vagina. As the device 10 is fully withdrawn, the second suture 180 still extends through the fistula (and the vagina and rectum). With the device 10 further withdrawn from the patient, the implantable material eventually enters the rectum and then the fistula. Specifically, the distal end 106 of the elongate portion 104 first enters the fistula, with a portion running fully through the fistula and entering the vagina.

With continued tension on the second suture 180, the implantable material 100 is fully drawn through the fistula tract until the button 110 contacts the rectal mucosa proximate the fistula. The button 110 is configured with a diameter substantially larger than the fistula to prevent the button 110 from entering the fistula. When the physician feels the tension developed in the second suture 180 as the button 110 contacts the rectal mucosa proximate the fistula, the physician receives tactile notification that the implantable material 100, and specifically the elongate portion 104 is properly positioned within the fistula tract. The physician then sews the button 110 to the rectal mucosa, while maintaining traction upon the second suture 180 (and therefore the elongate portion 104 of the implantable material 100), to retain the proper positioning of the implantable material 100 with respect to the rectal mucosa and the fistula. The physician places one or more stitches between the button 110 and the rectal mucosa, and uses the one or more apertures 112 in the button 110, when provided.

After fixing the button 110 to the rectal mucosa, the physician next sews or otherwise fixes a portion of the elongate portion 104 of the implantable material 100 extending from the fistula to the vaginal mucosa proximate the fistula with one or more sutures. After the elongate portion 104 is fixed to the vaginal mucosa, any extra length of the elongate portion 104 extending beyond the portion fixed to the vaginal mucosa and the second suture 180 are each removed and discarded.

While the preferred embodiments of the disclosure have been described, it should be understood that the invention is not so limited and modifications may be made without departing from the invention. The scope of the invention is defined by the appended claims, and all devices that come within the meaning of the claims, either literally or by equivalence, are intended to be embraced therein.

What is claimed is:

1. A medical device comprising:
an elongate member extending between a first end portion and a second end portion;
a brush disposed upon the elongate member between the first end portion and the second end portion;
a first aperture disposed proximate the first end portion and a second aperture disposed proximate the second end portion;
a first suture disposed through the first aperture and tied upon itself to form a first loop, and a second suture disposed through the second aperture and tied upon itself to form a second loop, wherein each of the first loop and the second loop are configured to receive an implantable material.

2. The medical device of claim 1, wherein the elongate member is tubular and defines a lumen therethrough.

3. The medical device of claim 2, wherein the brush comprises a central portion with a plurality of radially extending bristles.

4. The medical device of claim 3, wherein the brush further comprises a wire with a central portion supporting the radially extending bristles and first and second extended ends disposed on opposite sides of the central portion that are each received within the lumen.

5. The medical device of claim 4, wherein the central wire comprises a plurality of wires twisted together along a length of the wire.

6. The medical device of claim 3, wherein the central portion is substantially shorter than each of the first end portion and the second end portion.

7. The medical device of claim 2, wherein the first end portion defines a first end and the second end portion defines a second end.

8. The medical device of claim 7, wherein one of the first end and the second end allows fluid communication with the lumen.

9. The medical device of claim 8, further comprising an aperture defined on a side surface of one of the first end portion or the second end portion.

10. The medical device of claim 7, wherein the first end and the second end are each substantially closed.

11. The medical device of claim 1, wherein the implantable material comprises a biomaterial.

12. The medical device of claim 11, wherein the biomaterial is SIS.

13. A medical device comprising:
an elongate member extending between a first end portion and a second end portion, the elongate member defining a lumen through at least a portion thereof;
a brush disposed upon the elongate member between the first end portion and the second end portion;
a first aperture defined in the first end portion and a second aperture defined in the second end portion;
a first suture disposed through the first aperture and tied to itself to define a first loop and a second suture disposed through the second aperture and tied to itself to define a second loop, wherein each of the first and second loops are configured to receive an implantable material.

14. The medical device of claim 13, wherein the brush comprises a central portion with a plurality of radially extending bristles and first and second ends disposed on opposite sides of the central portion that are each received with in the lumen.

15. The medical device of claim 14, wherein the brush further comprises a central wire that extends between the first and second ends and receives the plurality of radially extending bristles.

16. The medical device of claim 15, wherein the central wire comprises a plurality of wires woven together.

17. The medical device of claim 13, wherein the first end portion and the second end portion each extend substantially the same distance from the brush.

18. The medical device of claim 13, wherein the first end portion comprises a first end that includes a first end aperture that is configured to allow fluid communication with the lumen.

19. The medical device of claim 18, further comprising a radial aperture proximate the brush, the radial aperture in fluid communication with the lumen and the first end aperture.

20. The medical device of claim 13, wherein the elongate member is substantially flexible.
